(19) United States
(12) Patent Application Publication
(10) Pub. No.: US 2005/0032879 A1
(43) Pub. Date: Feb. 10, 2005

(54) Formulations and use of a beta-blocker and an ACE-inhibitor for the treatment of cardiovascular diseases

(76) Inventors: Temple Okarter, Montvale, NJ (US); Richard J. Aiena, Howell, NJ (US); Bhudev Sharma, Edison, NJ (US)

Correspondence Address: KRAMER & AMADO, P.C., 2001 JEFFERSON DAVIS HWY, SUITE 1101, ARLINGTON, VA, 22202, US

(21) Appl. No.: 10/635,443

(22) Filed: Aug. 7, 2003

(51) Int. Cl. A61K 31/401; A61K 31/137

(52) U.S. Cl. 514/423; 514/651

(57) ABSTRACT

The use of a beta-blocker and an ACE-inhibitor in combination for the treatment of cardiovascular disorders, including hypertension and congestive heart failure, is provided. In particular, pharmaceutical formulations which use metoprolol tartrate and enalapril maleate in a therapeutically effective combination in a single extended release dosing unit for the treatment of congestive heart failure are disclosed as well as methods of using those formulations, thereby resulting in a decrease in a patient's overall pill burden.

The present invention relates to the use of a beta-blocker and an ACE-inhibitor in combination for the treatment of hypertension and in particular to the treatment of congestive heart failure, and more particularly to the use of metoprolol tartrate or pharmaceutically acceptable salts thereof and enalapril maleate or pharmaceutically acceptable salts thereof in combination for the treatment of hypertension and congestive heart failure.

### BACKGROUND

Combination therapy affords the physician and the patient the opportunity to more effectively treat diseases that may stem from more than one cause. When used correctly and appropriately, combination therapy leads to better outcomes than monotherapy by treating more than one cause of the disease and/or by synergistically enhancing the action of one of the component drugs.

Compliance is a major problem in the management of many disease states. Patients taking a large number of medications often forget to take their medications or are unwilling to take their medications. In cardiovascular disease, however, a number of medications are required to keep patients from decompensating. For example, patients with congestive heart failure require a large number of medications to improve their symptoms and improve their survival chances. For improving survival in patients with congestive heart failure, angiotensin-converting-enzyme (ACE) inhibitors are used to reduce mortality. Beta-blockers are also used in treating congestive heart failure and other cardiovascular disorders. If these two classes of medications could be combined into a single dosage unit, that will help treat hypertension and congestive heart failure with the ease of using one tablet alone.

### SUMMARY OF THE INVENTION

The present invention has the advantage of combining an ACE-inhibitor with a beta-blocker in one tablet or dosage form. The present invention also has the advantage of a having a number of dosage combinations and ratios to allow for easy and rapid titration of medications.

In accordance with the present invention, there is provided a method of using a beta-blocker and an ACE-inhibitor in combination for the treatment of cardiovascular disorders, such as congestive heart failure or hypertension. The method includes administering to a subject patient with a cardiovascular disorder a single dosage unit which has a therapeutically effective combination of a beta-blocker and an ACE-inhibitor. In the method of the invention, the single dosage unit would be administered repeatedly on a periodic schedule, such as every eight, twelve, or twenty four hours. The beta-blocker is selected from acebutolol, acebutolol HCL, atenolol, betaxolol, betaxolol HCL, bisoprolol, bisoprolol hemifumarate, carteolol, carteolol HCL, carvedilol, esmolol, esmolol HCL, labetalol, labetalol HCL, metoprolol, metoprolol succinate, metoprolol tartrate, nadolol, penbutolol, penbutolol sulfate, pindolol, propranolol, propranolol HCL, sotalol, sotalol HCL, timolol and timolol hydrogen maleate salt, and their pharmaceutically effective salts. The ACE-inhibitor is selected from benazepril, benazepril HCL, benazeprilat, captopril, enalapril, enalapril maleate, enalaprilat, fosinopril, fosinopril sodium salt, lisinopril, moexipril, perindopril, perindopril tert-Butylamine, perindoprilat, quinapril, quinapril HCL, quinaprilat, ramipril, ramiprilat, trandolapril and trandolaprilat, and their pharmaceutically effective salts. The method preferably uses enalapril maleate as the ACE-inhibitor and metoprolol tartrate as the beta-blocker. A therapeutically effective combination includes from about 2.5 mg to about 60 mg of enalapril maleate and from about 20 mg to about 250 mg of metoprolol tartrate. Therapeutically effective combinations of metoprolol tartrate and enalapril maleate in a ratio from 2.5 to 1 to 100 to 1 may also be used with the method of the present invention. Preferably, the method uses a single dosage unit which is an extended release dosage unit, although immediate release dosage units may also be used.

In accordance with the present invention, there is provided an extended release dosage unit for use in the treatment of cardiovascular disorders, such as congestive heart failure and/or hypertension. The extended release dosage unit includes a core section and an outer section. The core section of the extended release dosage unit contains a therapeutically effective combination of metoprolol tartrate and enalapril maleate, while the outer section includes a pharmaceutically acceptable dissolvable coating. After the outer section dissolves, the extended release dosage unit releases the drug combination according to a designed dissolution profile. The extended release dosage unit includes a therapeutically effective combination of metoprolol tartrate and enalapril maleate in a ratio of 2.5 to 1 up to and including 100 to 1 of metoprolol tartrate to enalapril maleate, with ratios from 5 to 1 to 10 to 1 preferred. The amounts in milligrams are as noted above (2.5 to 60 mg for enalapril maleate and 20 to 250 mg for metoprolol tartrate). Immediate release dosage units are also within the scope of the invention.

The present invention also provides a method of reducing a patient's pill burden in the treatment of cardiovascular disorders, such as congestive heart failure and/or hypertension. The method includes the step of reducing the amount of pills a patient with a cardiovascular disorder ingests per dosing period by administering to the patient a therapeutically effective combination of metoprolol tartrate and enalapril maleate in a single extended release dosage unit; and then repeating this step for each successive dosing period. According to the method, a therapeutically effective combination of metoprolol tartrate and enalapril maleate is from a 2.5 to 1 ratio to a 100 to 1 ratio, with a 5 to 1 ratio to a 10 to 1 ratio of metoprolol tartrate to enalapril maleate being preferred. The amounts in milligrams are as noted above.

In another embodiment of the present invention there is provided a single dosage unit for use in the treatment of cardiovascular disorders which has a core section and a pharmaceutically acceptable outer section. The core section contains a plurality of beads with each of the beads having a drug-containing section. The plurality of beads includes a first type of bead which contains metoprolol tartrate in the drug-containing section and a second type of bead which contains enalapril maleate in the drug-containing section. Together, the first type of bead and the second type of bead form a therapeutically effective combination of metoprolol tartrate and enalapril maleate. The amounts and ratios of metoprolol tartrate and enalapril maleate are as noted above. Preferably, at least some of the first or second type of beads may include a drug release coating which at least substantially envelops the drug-containing section of the beads. This coating can be modified to provide a desired dissolution profile.

In another embodiment of the invention and which is suitable for an immediate release product, powders containing the ACE-inhibitor and the beta-blocker are mixed together in the dosage unit. The powder mixture contains an ACE-inhibitor and a beta-blocker from the list noted above and in the ratios shown above. The mixture also contains other known ingredients for making the appropriate dosage form with desirable release profiles. The dosage form may or may not be coated. The coating may or may not contain any or both of the ACE-inhibitor and beta-blocker. The mixture of powders may contain beads of the active ingredient(s).

The combination of the ACE-inhibitor and beta-blocker, in particular enalapril maleate and metoprolol tartrate or their pharmaceutically effective salts, in a single dosage unit for treating cardiovascular disorders has the unexpected advantages of reducing the overall pill burden by reducing the total dosage amount. The metabolic effects are increased and the negative side effects are decreased by having the therapeutically effective combination in a single dosage unit. In sum, there is increased efficacy. It is believed that this is due to the reduced toxicity from a single dosage unit with a therapeutically effective combination versus separate individual doses of enalapril and metoprolol. In this manner, the overall dosage may be reduced as less pills provide equivalent therapeutic effectiveness.

These and other embodiments of the present invention are described in the detailed description which follows.

### BRIEF DESCRIPTION OF THE DRAWINGS

FIG. 1 is a cross-sectional diagram of a single dosage unit of the present invention.

FIG. 2 is a cross-sectional diagram of a single dosage unit of the present invention showing an alternative embodiment which includes a number of release beads in the core of the dosage unit.

### DETAILED DESCRIPTION

By combining an ACE-inhibitor and a beta-blocker in a single dosage unit, patients who suffer cardiovascular disorders would have a significantly reduced pill burden. With the present invention, the number of pills a patient is required to ingest on a daily basis is reduced from 7-8 pills per day to 2-3 pills per day. In accordance with the present invention, there is provided a method of using a beta-blocker and an ACE-inhibitor in combination for the treatment of cardiovascular disorders, such as congestive heart failure or hypertension. The method includes administering to a subject patient with a cardiovascular disorder a single dosage unit which has a therapeutically effective combination of a beta-blocker and an ACE-inhibitor. In the method of the invention, the single dosage unit would be repeatedly administered on a periodic schedule, such as every eight, twelve, or twenty four hours. The beta-blocker is selected from acebutolol, acebutolol HCL, atenolol, betaxolol, betaxolol HCL, bisoprolol, bisoprolol hemifumarate, carteolol, carteolol HCL, carvedilol, esmolol, esmolol HCL, labetalol, labetalol HCL, metoprolol, metoprolol succinate, metoprolol tartrate, nadolol, penbutolol, penbutolol sulfate, pindolol, propranolol, propranolol HCL, sotalol, sotalol HCL, timolol and timolol hydrogen maleate salt, and their pharmaceutically effective salts. The ACE-inhibitor is selected from benazepril, benazepril HCL, benazeprilat, captopril, enalapril, enalapril maleate, enalaprilat, fosinopril, fosinopril sodium salt, lisinopril, moexipril, perindopril, perindopril tert-Butylamine, perindoprilat, quinapril, quinapril HCL, quinaprilat, ramipril, ramiprilat, trandolapril and trandolaprilat, and their pharmaceutically effective salts. The method preferably uses enalapril maleate as the ACE-inhibitor and metoprolol tartrate as the beta-blocker. A therapeutically effective combination includes about 2.5 mg to about 60 mg of enalapril maleate and about 20 mg to about 250 mg of metoprolol tartrate. Therapeutically effective combinations of metoprolol tartrate and enalapril maleate in ratios of 2.5 to 1 up to and including 100 to 1, and preferably from 5 to 1 to 10 to 1, may also be used with the method of the present invention. Preferably, the method uses a single dosage unit which releases the drug combination of metoprolol tartrate and enalapril maleate according to a designed dissolution profile. Typically, the dissolution profile is in 0.1 N HCl and/or pH 6.8 buffer solution and is measured according to U.S. Pharmacopeia XXV using apparatus 2 paddle assembly at 100 r.p.m., 37° C. or may be modified as necessary to distinguish the specific combination.

The present invention also provides a method of reducing a patient's pill burden in the treatment of cardiovascular disorders, such as congestive heart failure and/or hypertension. The method includes the step of reducing the amount of pills a patient with a cardiovascular disorder ingests per dosing period by administering to the patient a therapeutically effective combination of metoprolol tartrate and enalapril maleate in a single extended release dosage unit; and then repeating this step for each successive dosing period. The single extended release dosage unit in this method provides a dissolution profile as noted above. According to the method, a therapeutically effective combination of metoprolol tartrate and enalapril maleate is from 2.5 to 1 ratio to 100 to 1 ratio, and preferably from a 5 to 1 ratio to a 10 to 1 ratio of metoprolol tartrate to enalapril maleate. Each of these compounds is described in detail below.

### Enalapril Maleate

Enalapril maleate, C₂₀H₂₈N₂O₅.C₄H₄O₄ is a white to off-white, crystalline powder with a chemical structure:

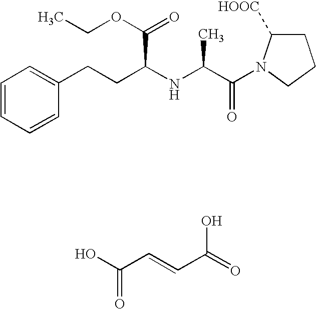

Enalapril is a pro-drug. It undergoes hydrolysis to form enalaprilat which is the active Angiotensin Converting Enzyme (ACE) inhibitor in both human and animal subjects. ACE-inhibitors block the conversion of angiotensin I to angiotensin II. Angiotensin II is a vasoconstrictor and stimulator of aldosterone secretion by the adrenal cortex. The beneficial effects of enalapril in hypertension and heart failure result from the suppression of the rennin-angiotensin-aldosterone system.

### Metoprolol Tartrate

Metoprolol tartrate (C₁₅H₂₅NO₃)₂.C₄H₆O₆ is a white practically odorless crystalline powder with a chemical structure:

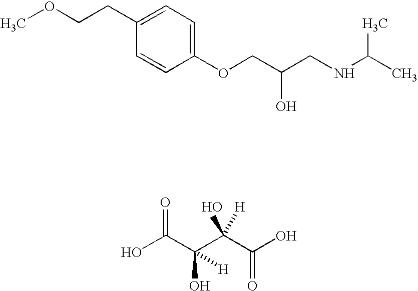

Metoprolol tartrate is a beta-adrenergic receptor blocking agent with preferential blockage of beta₁ receptors located chiefly in cardiac muscle. It also blocks the beta₂ receptor primarily located in bronchial and vascular musculature. The beta₁ blockade by metoprolol reduces heart rate and cardiac output at rest and exercise. It also reduces systolic blood pressure upon exercise as well as isoproterenol-induced tachycardia or reflex orthostatic tachycardia. Animal and human experiments indicate that metoprolol slows the sinus rate and decreases AV nodal conduction possibly due in part to the competitive antagonism of cathecholamines at peripheral (especially cardiac) adrenergic neuron sites resulting in a decrease in cardiac output. Lopressor® Package Insert, Revision Novartis, East Hanover, N.J. April (1999) and Metoprolol Tartrate product information, Mylan Pharmaceuticals Inc., Jan. (2002). Since the blockade of the renin-Angiotensin system with ACE-inhibitors has been established as the standard treatment in heart failure, and since the sympathetic nervous system is often activated prior to the renin-Angiotensin system in heart failure, blockade of the sympathetic nervous system should be complementary to the effects of ACE inhibition. Sharpe, Norman: Heart Failure Management, Martin Duniz, (2002), pp. 108-111.

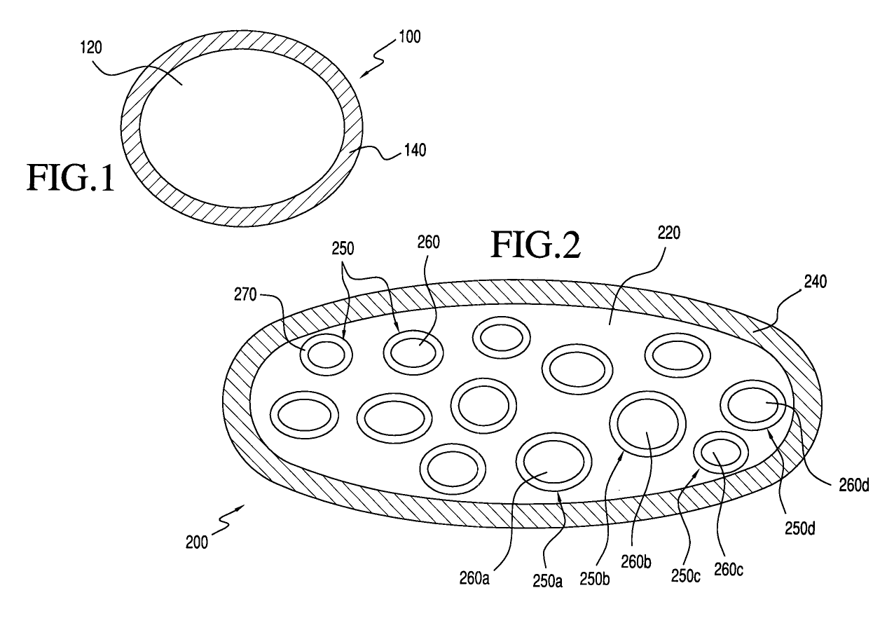

Referring to FIG. 1, there is shown the cross section of a single dosage unit 100 of the present invention which is used in the treatment of cardiovascular disorders such as congestive heart failure and hypertension. The extended release dosage unit 100 comprises a core section 120 and an outer section 140. The core section 120 contains a therapeutically effective combination of metoprolol tartrate and enalapril maleate. The outer section 140 is comprised of a pharmaceutically acceptable dissolvable coating which releases the contents of the core section 120 over a particular time period, (such as eight, twelve, or twenty four hours, etc.), when the unit 100 is ingested by a patient to provide an in vivo profile. In this manner, the metoprolol tartrate and enalapril maleate are combined in a single dosage unit 100, thereby reducing the amount of pills a patient with a particular cardiovascular disorder ingests per dosing period, ie. the patient's pill burden.

The dosage unit 100 may be an extended release tablet or capsule. Therapeutically effective amounts of enalapril maleate are preferably from about 2.5 mg to about 60 mg. Therapeutically effective amounts of metoprolol tartrate are preferably from about 20 mg to about 250 mg. Within the embodiments of the present invention, particular combinations containing 25/2.5; 25/5; 25/10; 50/5; 50/10; and 100 mg/20 mg of metoprolol tartrate and enalapril maleate, respectively, in extended release oral tablets and/or capsules are preferred. The ratio of metoprolol tartrate to enalapril maleate is preferably from 5 to 1 up to and including 10 to 1, although ratios as low as 2.5 to 1 and as high as 100 to 1 are included in the invention. The products are manufactured by processes and use excipients commonly known in the art for oral solid dosage forms. Examples of some excipients include lactose, starch, magnesium stearate, cellulose derivatives, and acceptable dyes and pigments. The excipients would be used to form the pharmaceutically acceptable dissolvable coating on outer section 140 of the single dosage unit 100. Those skilled in the art would recognize that FIG. 1 alternatively describes an immediate release oral dosage form of the present invention with the outer section 140 dissolving immediately upon ingestion to release the drug combination of metoprolol tartrate and enalapril maleate from the core 120. The drug combinations in this embodiment are in the amounts and ratios described above.

In the present invention, the drug release of the metoprolol tartrate and enalapril maleate can be controlled and modified by use of various coatings, such as polymeric membrane coatings, to release the drugs according to various release profiles. For example, the release profile may be pH dependent, pH independent, enteric coatings, and with or without delay time. Examples of polymeric membrane coatings include hydroxypropylmethyl cellulose, hydroxypropyl cellulose, hydroxypropylmethyl cellulose phthalate (e.g. HP 50, HP 55), ethyl cellulose, cellulose acetate phthalate, and the Eudragit® film coating materials, such as Eudragit® RL, Eudragit® RS, Eudragit® L, S, E, and NE.

Ethyl cellulose can be used alone or in a combination with various water soluble polymers such as hydroxypropyl or methyl cellulose to adjust the permeability of the coating. Other water soluble polymers include polyethylene glycol, polyvinyl pyrrolidone, etc. Povidone K 30 or Povidone K 90 are suitable grade film formers and release controlling agents.

Pigments and/or plasticizers can be added to the polymeric coatings to improve and modify the technical properties or alter the release characteristics of the membrane. Examples of suitable plasticizers are polyethylene glycols, propylene glycol, glycerides, citrate esters, tributyl citrate, triethyl citrate, acetyl tributyl citrate, acetal triethyl citrate.

The Eudragit® film coating materials are produced by Rohm Pharma Polymers (Degussa) and have an acrylic resin basis. For example, the Eudragit RL and RS materials are copolymers synthesized from acrylic and methacrylic acid esters with a low content of quaternary ammonium groups. The molar ratio of these ammonium groups to the remaining neutral (meth)acrylic acid esters is 1:20 for Eudragit RL and 1:40 for Eudragit RS in order to provide different permeability characteristics.

Referring now to FIG. 2, there is shown an alternative embodiment of the present invention. In this embodiment, there is a single dosage unit 200 which has a core section 220 and an outer section 240. The outer section 240 is comprised of pharmaceutically acceptable excipients known in the art. The core section 220 contains a plurality of beads 250, such as beads numbered as 250(a)-(d). Each of the beads 250 have a drug-containing section 260, such as drug-containing sections numbered as 260(a)-(d). Each of the beads may also have a drug release coating 270 enveloping or substantially enveloping the drug-containing section 260 of the beads 250. The totality of the beads 250 within the core 220 contain a therapeutically effective combination of metoprolol tartrate and enalapril maleate in the amounts, ratios and combinations as described above with respect to FIG. 1.

The drug containing section 260 of an individual bead 250 may contain metoprolol tartrate and enalapril maleate in combination, i.e. each of beads 250(a)-(d) contain a combination of metoprolol tartrate and enalapril maleate in the respective drug containing sections 260(a)-260(d). Alternatively, the drug containing section 260 of an individual bead 250 may include only a single compound of either metoprolol tartrate or enalapril maleate. For example, beads 250(a) and 250(b) contain metoprolol tartrate in the respective drug containing sections 260(a) and 260(b) while beads 250(c) and 250(d) contain enalapril maleate in respective drug containing sections 260(c) and 260(d), thereby forming a mixture or blend of metoprolol tartrate beads 250(a) and 250(b) with enalapril maleate beads 250(c) and 250(d) within the core 220.

The drug containing section 260 of the beads 250 is prepared according to any of a number of methods known in the art, such as binding the drug compounds of enalapril maleate and metoprolol tartrate to an inert carrier with a conventional binding agent. The enalapril maleate and metoprolol tartrate may be bound separately. The inert carrier is typically a starch or sugar sphere, but any pharmaceutically acceptable inert carrier may be utilized. Conventional excipients may be added to the drug compounds to expedite handling or improve properties.

The drug release coating 270 which may envelop or substantially envelop the drug containing sections 260 of the beads 250 can be formed from the various polymeric coatings described previously. Therefore, the drug release of metoprolol tartrate and enalapril maleate can be modified and controlled to release the drugs according to various release profiles. An identical drug release coating 270 may be applied across all of the beads 250 or a different drug release coating 270 may be applied to different beads 250. For example, a short release or immediate release bead may be formed by applying a smaller amount or no coating to a first group of beads. A long or delayed release bead may be formed by applying a differing, larger amount of drug release coating 270 to a second group of beads. Additionally, beads containing enalapril maleate may have a particular amount of coating to produce a desired enalapril maleate dissolution profile in a patient, while the beads containing metoprolol tartrate may have a second amount of coating to produce the desired metoprolol tartrate dissolution profile in the patient. Different coating materials may be used as well. In this manner, a therapeutically effective combination of metoprolol tartrate and enalapril maleate may be delivered in a desired amount over a desired periodic interval through a single dosing unit.

Similar to above, therapeutically effective amounts of enalapril maleate are preferably from about 2.5 mg to about 60 mg. Therapeutically effective amounts of metoprolol tartrate are preferably from about 20 mg to about 250 mg. Particular combinations containing 25/2.5; 25/5; 25/10; 50/5; 50/10; and 100 mg/20 mg of metoprolol tartrate and enalapril maleate, respectively, in extended release oral tablets and/or capsules are preferred.

The drug release coatings 270 may be applied to the drug containing section 260 using methods and techniques known in the art. Typically suspensions, emulsions, or solutions of the coating materials are prepared as known in the art and then applying the fluid by known coating techniques, (e.g., fluid bed coaters, pan coaters, etc.). After coating, the beads are dried as desired, and then the amounts of each type of bead (immediate, short, delayed or long, enalapril maleate only, metoprolol tartrate only, etc.) are selected for mixture into a final single dosage form or unit, such as a gelatin capsule.

A non-limiting example of a preparation of an embodiment of the present invention is provided below:

In this example, a single dosage unit with a combination of 25 mg of metoprolol tartrate and 10 mg enalapril maleate is prepared.

A. Preparation of Metoprolol Tartrate Coated Beads

The following ingredients and amounts are used:

| | | |
|---|---|---|
| 1.) Metoprolol tartrate | 25 mg | Active ingredient |
| 2.) Lactose | 25 mg | Filler |
| 3.) Microcrystalline Cellulose | 10 mg | Filler |
| 4.) Hydroxypropyl Methylcellulose, CR | 125 mg | Release regulating |
| | | agent |
| 5.) Povidone | 10 mg | Binder/Release |
| | | regulating agent |
| 6.) Water, Purified | qs | Solvent. Removed |
| | | during drying |

a.) Mix ingredients 1 through 4 together.
b.) Granulate with Povidone, adding the purified water, if necessary and granulate to achieve the desired granulation.
c.) Dry the granulation.
d.) Size the granules.
e.) Coat to form beads using Coating Solution A below or similar coating solution.

Alternatively,
a) Mix ingredients 1 through 4
b) Coat to form beads using Coating Solution B below or similar coating solution.

Coat granules using formula below:

Coating Solution A:

| | | |
|---|---|---|
| 7. Hydroxypropyl Methylcellulose [HPMC] | 5% | Film Former |
| 8. Plasticizer e.g. Propylene Glycol, PEG 400 | 0.5-5% | Plasticizer |
| 9. Water, Purified qs | 100% | Solvent |

Coating Solution B:

| | | | |
|---|---|---|---|
| 10. | Eudragit RS 30D or | 20% | Film former |
| | suitable viscosity grade HPMC | | |
| 11. | Plasticizer, e.g. Triacetin, PEG 400 | 0.5-5% | Plasticizer |
| 12. | Water, Purified qs | 100% | Solvent |

Note: Colorant may be added to granulation or coating solution.

B. Preparation of Enalapril Maleate

The following ingredients and amounts are used:

| | | |
|---|---|---|
| Enalapril maleate | 10 mg | Active |
| Starch | 30 mg | Filler |
| Microcrystalline Cellulose | 30 mg | Filler |
| Disintegrant, e.g. PVP XL, Sodium | 5 mg | Disintegrant |
| starch glycolate | | |
| Magnesium Stearate | 5 mg | Lubricant |

Mix each of these ingredients together and then combine with the coated metoprolol tartrate beads from Step A above to obtain a mixture containing 25 mg of metoprolol tartrate and 10 mg enalapril maleate. This mixture is then compressed into tablets. This provides a single dosage unit which contains an immediate release of enalapril maleate and a sustained or extended release of metoprolol tartrate. The tablets may be coated with Coating Solution A above or a modified formula, with or without colorant as desired. This single dosage unit is then administered to a patient with a cardiovascular disease on a periodic basis to deliver a therapeutically effective combination of metoprolol tartrate and enalapril maleate, and thereby reduce the amount of pills a patient ingests in a particular dosing period.

Those skilled in the art would recognize that other combinations can be made in similar fashion by multiples of the above ingredients—for example a 50 mg metoprolol tartrate/10 mg enalapril maleate combination would be made by doubling the amounts of ingredients in Step A. Coating solutions may also be applied to the enalapril maleate preparation in Step B. Capsules may also be used.

The combination of the ACE-inhibitor and beta-blocker, in particular enalapril maleate and metoprolol tartrate or their pharmaceutically effective salts, in a single dosage unit for treating cardiovascular disorders has the unexpected advantages of reducing the overall pill burden by reducing the total dosage amount. The metabolic effects are increased and the negative side effects are decreased by having the therapeutically effective combination in a single dosage unit. In sum, there is increased efficacy. It is believed that this is due to the reduced toxicity from a single dosage unit with a therapeutically effective combination versus separate individual doses of enalapril and metoprolol. In this manner, the overall dosage may be reduced as less pills provide equivalent therapeutic effectiveness.

Although the present invention has been described in detail with particular reference to preferred embodiments thereof, it should be understood that the invention is capable of other different embodiments, and its details are capable of modifications in various obvious respects. As is readily apparent to those skilled in the art, variations and modifications can be effected while remaining within the spirit and scope of the invention. Accordingly, the foregoing disclosure, description, and figures are for illustrative purposes only, and do not in any way limit the invention, which is defined only by the claims.

1. A method of using a beta-blocker and an ACE-inhibitor in combination for the treatment of cardiovascular disorders comprising:
administering to a subject with a cardiovascular disorder a single dosage unit,
said single dosage unit having a therapeutically effective combination of a beta-blocker and an ACE-inhibitor;
repeating said administering step on a periodic schedule.

2. The method according to claim 1 wherein said ACE-inhibitor is enalapril maleate or pharmaceutically acceptable salts thereof.

3. The method according to claim 2 wherein said beta-blocker is metoprolol tartrate or pharmaceutically acceptable salts thereof.

4. The method according to claim 3 wherein said therapeutically effective combination includes about 2.5 mg to about 60 mg of enalapril maleate.

5. The method according to claim 3 wherein said therapeutically effective combination includes about 20 mg to about 250 mg of metoprolol tartrate.

6. The method according to claim 3 wherein said therapeutically effective combination is metoprolol tartrate and enalapril maleate in a ratio from 2.5 to 1 to 100 to 1.

7. The method according to claim 6 wherein said therapeutically effective combination is metoprolol tartrate and enalapril maleate in a ratio from 5 to 1 to 10 to 1.

8. The method according to claim 1 wherein said single dosage unit is an extended release dosage unit.

9. The method according to claim 1 wherein said cardiovascular disorder is congestive heart failure.

10. A method of using metoprolol tartrate or pharmaceutically acceptable salts thereof and enalapril maleate or pharmaceutically acceptable salts thereof in combination for the treatment of cardiovascular disorders comprising:
administering to a subject with a cardiovascular disorder a single dosage unit having a therapeutically effective combination of metoprolol tartrate or pharmaceutically acceptable salts thereof and enalapril maleate or pharmaceutically acceptable salts thereof;
repeating said administering step on a periodic schedule.

11. The method according to claim 10 wherein said therapeutically effective combination includes about 2.5 mg to about 60 mg of enalapril maleate.

12. The method according to claim 10 wherein said therapeutically effective combination of includes about 20 mg to about 250 mg of metoprolol tartrate.

13. The method according to claim 10 wherein said cardiovascular disorder is congestive heart failure.

14. The method according to claim 10 wherein said single dosage unit is an extended release dosage unit.

15. An extended release dosage unit for use in the treatment of cardiovascular disorders comprising a core section and an outer section;
said core section containing a therapeutically effective combination of metoprolol tartrate or pharmaceutically acceptable salts thereof and enalapril maleate or pharmaceutically acceptable salts thereof;
said outer section comprising a pharmaceutically acceptable dissolvable coating.

16. The extended release dosage unit according to claim 15 wherein said therapeutically effective combination of metoprolol tartrate and enalapril maleate is from a 2.5 to 1 ratio to a 100 to 1 ratio of metoprolol tartrate to enalapril maleate.

17. The extended release dosage unit according to claim 16 wherein said therapeutically effective combination of metoprolol tartrate and enalapril maleate is from a 5 to 1 ratio to a 10 to 1 ratio of metoprolol tartrate to enalapril maleate.

18. The extended release dosage unit according to claim 15 wherein said therapeutically effective combination includes about 2.5 mg to about 60 mg of enalapril maleate.

19. The extended release dosage unit according to claim 15 wherein said therapeutically effective combination includes about 20 mg to about 250 mg of metoprolol tartrate.

20. A method of reducing a patient's pill burden in the treatment of cardiovascular disorders comprising:
reducing the amount of pills a patient with a cardiovascular disorder ingests per dosing period by administering to said patient a therapeutically effective combination of metoprolol tartrate or pharmaceutically acceptable salts thereof and enalapril maleate or pharmaceutically acceptable salts thereof in a single dosage unit; and
repeating said reducing step.

21. The method according to claim 20 wherein said single dosage unit is an extended release dosage unit.

22. The method according to claim 20 wherein said therapeutically effective combination of metoprolol tartrate and enalapril maleate is from a 2.5 to 1 ratio to a 100 to 1 ratio of metoprolol tartrate to enalapril maleate.

23. The method according to claim 22 wherein said therapeutically effective combination of metoprolol tartrate and enalapril maleate is from a 5 to 1 ratio to a 10 to 1 ratio of metoprolol tartrate to enalapril maleate.

24. The method according to claim 20 wherein said therapeutically effective combination includes about 2.5 mg to about 60 mg of enalapril maleate.

25. The method according to claim 20 wherein said therapeutically effective combination includes about 20 mg to about 250 mg of metoprolol tartrate.

26. A single dosage unit for use in the treatment of cardiovascular disorders comprising a core section and a pharmaceutically acceptable outer section;
said core section containing a plurality of beads with each of said beads having a drug-containing section;
said plurality of beads comprising a first type of beads which contain metoprolol tartrate or pharmaceutically acceptable salts thereof in said drug-containing section and a second type of beads which contain enalapril maleate or pharmaceutically acceptable salts thereof in said drug-containing section;
wherein said first type of beads and said second type of beads of said plurality of beads together form a therapeutically effective combination of metoprolol tartrate and enalapril maleate.

27. The single dosage unit according to claim 26 wherein at least some of said first type of beads have a drug release coating at least substantially enveloping said drug-containing section of said first type of beads.

28. The single dosage unit according to claim 26 wherein at least some of said second type of beads have a drug release coating at least substantially enveloping said drug-containing section of said second type of beads.

29. The single dosage unit according to claim 26 wherein said therapeutically effective combination of metoprolol tartrate and enalapril maleate is from a 2.5 to 1 ratio to a 100 to 1 ratio of metoprolol tartrate to enalapril maleate.

30. The single dosage unit according to claim 29 wherein said therapeutically effective combination of metoprolol tartrate and enalapril maleate is from a 5 to 1 ratio to a 10 to 1 ratio of metoprolol tartrate to enalapril maleate.

31. The single dosage unit according to claim 26 wherein said therapeutically effective combination includes about 2.5 mg to about 60 mg of enalapril maleate.

32. The single dosage unit according to claim 26 wherein said therapeutically effective combination includes about 20 mg to about 250 mg of metoprolol tartrate.

33. The single dosage unit according to claim 26 wherein said first type of beads further include enalapril maleate in said drug containing section.

34. The single dosage unit according to claim 26 wherein said second type of beads further include metoprolol tartrate in said drug containing section.

35. The method according to claim 10 wherein said single dosage unit comprises a core section and a pharmaceutically acceptable outer section;
said core section containing a plurality of beads with each of said beads having a drug-containing section;
said plurality of beads comprising a first type of beads which contain metoprolol tartrate in said drug-containing section and a second type of beads which contain enalapril maleate in said drug-containing section;
wherein said first type of beads and said second type of beads of said plurality of beads together form said therapeutically effective combination of metoprolol tartrate and enalapril maleate.

36. The method according to claim 1 wherein said single dosage unit is an immediate release dosage unit.
